(19) United States
(12) Patent Application Publication
(10) Pub. No.: US 2009/0259054 A1
(43) Pub. Date: Oct. 15, 2009

(54) Methods and Compositions for the Treatment of Angiogenesis and Macular Degeneration

(75) Inventors: Mingyao Liu, Pearland, TX (US); Tingfang Yi, Houston, TX (US); Zhengfang Yi, Houston, TX (US); Sung-Gook Cho, Houston, TX (US)

Correspondence Address: MEDLEN & CARROLL, LLP, 101 HOWARD STREET, SUITE 350, SAN FRANCISCO, CA, 94105, US

(73) Assignee: The Texas A&M University System

(21) Appl. No.: 12/359,910

(22) Filed: Jan. 26, 2009

Related U.S. Application Data
(60) Provisional application No. 61/023,628, filed on Jan. 25, 2008.

(51) Int. Cl. C07D 311/78

(52) U.S. Cl. 549/381

(57) ABSTRACT

The present invention relates to methods and compositions for the treatment of angiogenesis and macular degeneration. In preferred embodiments, the invention relates to the field of eye health. In some embodiments, the invention relates to the prevention and treatment of angiogenesis by administering compounds disclosed herein. In further embodiments, the invention relates to the prevention and treatment of macular degeneration by administering compounds disclosed herein. In still further embodiments, the invention relates to methods and compositions comprising gambogic acid and gambogic acid derivatives.

### STATEMENT OF GOVERNMENT SUPPORT

This invention was made in part with government support under grant number 1R01CA106479, from the National Institutes of Health. As such, the United States government has certain rights to the invention.

### FIELD OF THE INVENTION

The present invention relates to methods and compositions for the treatment of angiogenesis and macular degeneration. In preferred embodiments, the invention relates to the field of eye health. In some embodiments, the invention relates to the prevention and treatment of angiogenesis by administering compounds disclosed herein. In further embodiments, the invention relates to the prevention and treatment of macular degeneration by administering compounds disclosed herein. In still further embodiments, the invention relates to methods and compositions comprising gambogic acid and gambogic acid derivatives.

### BACKGROUND OF THE INVENTION

Angiogenesis is crucial for organ growth and repair; however, an imbalance in this process contributes to numerous diseases. Excessive angiogenesis leads to diseases and disorders such as inflammation, rheumatoid arthritis, psoriasis, diabetic retinopathy, impaired wound healing, cancer and macular degeneration. Inhibiting angiogenesis is a promising strategy for treatment of many diseases. Thus, there is a need to identity agents that prevent both angiogenesis and macular degeneration.

### SUMMARY OF THE INVENTION

The present invention relates to methods and compositions for the treatment of angiogenesis and macular degeneration. In preferred embodiments, the invention relates to the field of eye health. In some embodiments, the invention relates to the prevention and treatment of angiogenesis by administering compounds disclosed herein. In further embodiments, the invention relates to the prevention and treatment of macular degeneration by administering compounds disclosed herein. In still further embodiments, the invention relates to methods and compositions comprising gambogic acid and gambogic acid derivatives.

We demonstrate (below) the previously unreported inhibition of GA on HUVEC cell proliferation, migration, and tube formation, as well as the anti-angiogenesis activity of GA in vitro and in vivo. Our data suggest that compounds of the xanthone family as anti-angiogenesis and anti-cancer drugs.

In some embodiments, the invention relates to a method of treating macular degeneration comprising: providing: a subject diagnosed with macular degeneration, and a composition comprising gambogic acid or a gambogic acid derivative; and administering said compound to said subject. In further embodiments, said gambogic acid derivative is selected from the group consisting of methyl gambogate; 9,10-dihydrogambogic acid; 9,10-dihydrogambogyl (4-methylpiperazine); 9,10-dihydro-gambogyl (2-dimethylaminoethylamine); gambogyl diethylamine; gambogyl dimethyl-amine; gambogyl amine; gambogyl hydroxyamine; gambogyl piperidine; 6-methoxy-gambogic acid; 6-(2-dimethylaminoethoxy)-gambogic acid; 6-(2-piperidinylethoxy)-gambogic acid; 6-(2-morpholinylethoxy)-gambogic acid; 6-methoxy-gambogyl piperidine; gambogyl morpholine; gambogyl (2-dimethylaminoethylamine); 10-morpholinyl-gambogyl morpholine; 10-morpholinyl-gambogyl piperidine; 10-(4-methylpiperazinyl)-gambogyl piperidine; 10-(4-methylpiperazinyl)-gambogyl morpholine; 10-piperidinyl-gambogyl piperidine; 10-(4-methylpiperazinyl)-gambogyl (4-methylpiperazine); gambogyl (4-methylpiperazine); methyl-6-methoxy-gambogate; gambogenic acid; gambogenin; 10-methoxy-gambogic acid; 10-butylthio-gambogic acid; 10-(4-methylpiperazinyl)-gambogic acid; 10-pyrrolidinyl-gambogic acid; methyl-10-morpholinyl-gambogate; 10-piperidinyl-gambogic acid; 10-morpholinyl-gambogic acid; N-(2-gambogylamidoethyl)biotinamide; gambogyl (2-morpholinylethylamine); 9,10-epoxygambogic acid; gambogyl (4-(2-pyridyl)piperazine); 10-(4-(2-pyridyl)-piperazinyl)gambogyl (4-(2-pyridyl)piperazine); 6-acetylgambogic acid; 10-(4-(2-pyridyl)piperazinyl)gambogic acid; N-hydroxysuccinimidyl gambogate; 8-(gambogylamido)octanoic acid; 6-(gambogylamido)hexanoic acid; 12-(gambogylamido)dodecanoic acid; N-hydroxysuccinimidyl-8-(gambogylamido)-octanoate; N-hydroxysuccinimidyl-6-(gambogylamido)hexanoate; N-hydroxy-succinimidyl-12-(gambogylamido)dodecanoate; 10-methoxy-gambogyl piperidine; gambogyl (4-(2-pyrimidyl)piperazine); gambogyl (bis(2-pyridylmethyl)amine); gambogyl (N-(3-pyridyl)-N-(2-hydroxybenzyl)amine); gambogyl (4-benzylpiperazine); gambogyl (4-(3,4-methylenedioxybenzyl)piperazine); gambogyl (N-methyl-5-(methylamino)-3-oxapentylamine); gambogyl (N-methyl-8-(methylamino)-3,6-dioxaoctylamine); gambogyl (N-ethyl-2-(ethylamino)ethylamine); Gambogyl (4-isopropylpiperazine); gambogyl (4-cyclopentylpiperazine); gambogyl (N-(2-oxo-2-ethoxyethyl)-(2-pyridyl)methylamine); gambogyl (2,5-dimethylpiperazine); gambogyl (3,5-dimethylpiperazine); gambogyl (4-(4-acetylphenyl)piperazine); gambogyl (4-ethoxycarbonylpiperazine); gambogyl (4-(2-oxo-2-pyrrolidylethyl)piperazine); gambogyl (4-(2-hydroxyethyl)piperazine); gambogyl (N-methyl-2-(methylamino)ethylamine); gambogyl (N-methyl-2-(benzylamino)ethylamine); gambogyl (N-methyl-(6-methyl-2-pyridyl)methylamine); gambogyl (N-ethyl-2-(2-pyridyl)ethylamine); gambogyl (N-methyl-(2-pyridyl)methylamine); gambogyl (N-methyl-4-(3-pyridyl)butylamine); gambogyl (bis(3-pyridylmethyl)amine); gambogyl (2,4-dimethyl-2-imidazoline); gambogyl (4-methyl-homopiperazine); gambogyl (4-(5-hydroxy-3-oxapentyl)piperazine); gambogyl (3-dimethylaminopyrrolidine); gambogyl ((2-furanyl)methylamine); gambogyl (2-hydroxy-1-methyl-2-phenylethylamine); gambogyl (3,4,5-trimethoxybenzylamine); gambogyl (2-(2-methoxyphenyl)ethylamine); gambogyl (2-methoxybenzylamine); gambogyl (3,4-methylenedioxybenzylamine); gambogyl (2-(2,5-dimethoxy-phenyl)ethylamine); gambogyl (2-(3-methoxyphenyl)ethylamine); gambogyl (3-(piperidinyl)propylamine); gambogyl (2-(piperidinyl)ethylamine); gambogyl (3,4-dimethoxybenzylamine); gambogyl ((2-tetrahydrofuranyl)methylamine); gambogyl ((N-ethyl-2-pyrrolidinyl)methylamine); gambogyl (2-diethylaminoethylamine); gambogyl (2,2-dimethyl-3-dimethylaminopropylamine); gambogyl ((N-ethoxycarbonyl-4-piperidinyl)amine); gambogyl (2-carbamylpyrrolidine); gambogyl (3-(homopiperidinyl)-propylamine); gambogyl ((N-benzyl-4-piperidinyl)amine); gambogyl (2-(4-methoxyphenyl)ethylamine); gambogyl (4-oxa-hex-5-enylamine); gambogyl (6-hydroxyhexylamine); gambogyl (2-(3,5-dimethoxyphenyl)ethylamine); gambogyl (3,5-dimethoxybenzylamine); and gambogyl (2-carbamyl-2-(4-hydroxyphenyl)ethylamine). In still further embodiments, said subject is a mammal. In additional embodiments, said composition is a solution. In some embodiments, the mode of said administration is selected from the group consisting of optical, oral, parenteral, mucosol, buccal, vaginal, rectal, sublingual, inhalation, insufflation, intravenous, intrathecal, subcutaneous and intramuscular.

In some embodiments, the invention relates to a method of treating angiogenesis comprising: providing: a subject diagnosed with or at risk for angiogenesis, and a composition comprising gambogic acid or a gambogic acid derivative; and administering said compound to said subject. In further embodiments, said gambogic acid derivative is selected from the group consisting of methyl gambogate; 9,10-dihydrogambogic acid; 9,10-dihydrogambogyl (4-methylpiperazine); 9,10-dihydrogambogyl (2-dimethylamino-ethylamine); gambogyl diethylamine; gambogyl dimethylamine; gambogyl amine; gambogyl hydroxyamine; gambogyl piperidine; 6-methoxy-gambogic acid; 6-(2-dimethylaminoethoxy)-gambogic acid; 6-(2-piperidinylethoxy)-gambogic acid; 6-(2-morpholinylethoxy)-gambogic acid; 6-methoxy-gambogyl piperidine; gambogyl morpholine; gambogyl (2-dimethylaminoethylamine); 10-morpholinyl-gambogyl morpholine; 10-morpholinyl-gambogyl piperidine; 10-(4-methylpiperazinyl)-gambogyl piperidine; 10-(4-methylpiperazinyl)-gambogyl morpholine; 10-piperidinyl-gambogyl piperidine; 10-(4-methylpiperazinyl)-gambogyl (4-methylpiperazine); gambogyl (4-methylpiperazine); methyl-6-methoxy-gambogate; gambogenic acid; gambogenin; 10-methoxy-gambogic acid; 10-butylthio-gambogic acid; 10-(4-methylpiperazinyl)-gambogic acid; 10-pyrrolidinyl-gambogic acid; methyl-10-morpholinyl-gambogate; 10-piperidinyl-gambogic acid; 10-morpholinyl-gambogic acid; N-(2-gambogylamidoethyl)-biotinamide; gambogyl (2-morpholinylethylamine); 9,10-epoxygambogic acid; gambogyl (4-(2-pyridyl)piperazine); 10-(4-(2-pyridyl)piperazinyl)gambogyl (4-(2-pyridyl)-piperazine); 6-acetylgambogic acid; 10-(4-(2-pyridyl)piperazinyl)gambogic acid; N-hydroxysuccinimidyl gambogate; 8-(gambogylamido)octanoic acid; 6-(gambogylamido)-hexanoic acid; 12-(gambogylamido)dodecanoic acid; N-hydroxysuccinimidyl-8-(gambogylamido)octanoate; N-hydroxysuccinimidyl-6-(gambogylamido)hexanoate; N-hydroxysuccinimidyl-12-(gambogylamido)dodecanoate; 10-methoxy-gambogyl piperidine; gambogyl (4-(2-pyrimidyl)piperazine); gambogyl (bis(2-pyridylmethyl)amine); gambogyl (N-(3-pyridyl)-N-(2-hydroxybenzyl)amine); gambogyl (4-benzylpiperazine); gambogyl (4-(3,4-methylenedioxybenzyl)piperazine); gambogyl (N-methyl-5-(methylamino)-3-oxapentylamine); gambogyl (N-methyl-8-(methylamino)-3,6-dioxaoctylamine); gambogyl (N-ethyl-2-(ethylamino)ethylamine); Gambogyl (4-isopropylpiperazine); gambogyl (4-cyclopentylpiperazine); gambogyl (N-(2-oxo-2-ethoxyethyl)-(2-pyridyl)methylamine); gambogyl (2,5-dimethylpiperazine); gambogyl (3,5-dimethylpiperazine); gambogyl (4-(4-acetylphenyl)piperazine); gambogyl (4-ethoxycarbonylpiperazine); gambogyl (4-(2-oxo-2-pyrrolidylethyl)piperazine); gambogyl (4-(2-hydroxyethyl)piperazine); gambogyl (N-methyl-2-(methylamino)ethylamine); gambogyl (N-methyl-2-(benzylamino)ethylamine); gambogyl (N-methyl-(6-methyl-2-pyridyl)methylamine); gambogyl (N-ethyl-2-(2-pyridyl)ethylamine); gambogyl (N-methyl-(2-pyridyl)methylamine); gambogyl (N-methyl-4-(3-pyridyl)butylamine); gambogyl (bis(3-pyridylmethyl)amine); gambogyl (2,4-dimethyl-2-imidazoline); gambogyl (4-methyl-homopiperazine); gambogyl (4-(5-hydroxy-3-oxapentyl)piperazine); gambogyl (3-dimethylaminopyrrolidine); gambogyl ((2-furanyl)methylamine); gambogyl (2-hydroxy-1-methyl-2-phenylethylamine); gambogyl (3,4,5-trimethoxybenzylamine); gambogyl (2-(2-methoxyphenyl)ethylamine); gambogyl (2-methoxybenzylamine); gambogyl (3,4-methylenedioxybenzylamine); gambogyl (2-(2,5-dimethoxyphenyl)-ethylamine); gambogyl (2-(3-methoxyphenyl)ethylamine); gambogyl (3-(piperidinyl)propylamine); gambogyl (2-(piperidinyl)ethylamine); gambogyl (3,4-dimethoxybenzylamine); gambogyl ((2-tetrahydrofuranyl)methylamine); gambogyl ((N-ethyl-2-pyrrolidinyl)methylamine); gambogyl (2-diethylaminoethylamine); gambogyl (2,2-dimethyl-3-dimethylaminopropylamine); gambogyl ((N-ethoxycarbonyl-4-piperidinyl)amine); gambogyl (2-carbamylpyrrolidine); gambogyl (3-(homopiperidinyl)-propylamine); gambogyl ((N-benzyl-4-piperidinyl)amine); gambogyl (2-(4-methoxyphenyl)ethylamine); gambogyl (4-oxa-hex-5-enylamine); gambogyl (6-hydroxyhexylamine); gambogyl (2-(3,5-dimethoxyphenyl)ethylamine); gambogyl (3,5-dimethoxybenzylamine); and gambogyl (2-carbamyl-2-(4-hydroxyphenyl)ethylamine). In still further embodiments, said subject is a mammal. In additional embodiments, said composition is a solution. In some embodiments, the mode of said administration is selected from the group consisting of optical, oral, parenteral, mucosol, buccal, vaginal, rectal, sublingual, inhalation, insufflation, intravenous, intrathecal, subcutaneous and intramuscular.

In some embodiments, the invention relates to a method for treating a disease characterized by angiogenesis comprising: providing: a subject diagnosed with or at risk for said disease characterized by angiogenesis, and a composition comprising gambogic acid or a gambogic acid derivative; and administering said compound to said subject. In further embodiments, said gambogic acid derivative is selected from the group consisting of methyl gambogate; 9,10-dihydrogambogic acid; 9,10-dihydrogambogyl (4-methylpiperazine); 9,10-dihydrogambogyl (2-dimethylaminoethylamine); gambogyl diethylamine; gambogyl dimethylamine; gambogyl amine; gambogyl hydroxyamine; gambogyl piperidine; 6-methoxy-gambogic acid; 6-(2-dimethylaminoethoxy)-gambogic acid; 6-(2-piperidinylethoxy)-gambogic acid; 6-(2-morpholinylethoxy)-gambogic acid; 6-methoxy-gambogyl piperidine; gambogyl morpholine; gambogyl (2-dimethylaminoethylamine); 10-morpholinyl-gambogyl morpholine; 10-morpholinyl-gambogyl piperidine; 10-(4-methylpiperazinyl)-gambogyl piperidine; 10-(4-methylpiperazinyl)-gambogyl morpholine; 10-piperidinyl-gambogyl piperidine; 10-(4-methylpiperazinyl)-gambogyl (4-methylpiperazine); gambogyl (4-methylpiperazine); methyl-6-methoxy-gambogate; gambogenic acid; gambogenin; 10-methoxy-gambogic acid; 10-butylthio-gambogic acid; 10-(4-methylpiperazinyl)-gambogic acid; 10-pyrrolidinyl-gambogic acid; methyl-10-morpholinyl-gambogate; 10-piperidinyl-gambogic acid; 10-morpholinyl-gambogic acid; N-(2-gambogylamidoethyl)biotinamide; gambogyl (2-morpholinylethylamine); 9,10-epoxygambogic acid; gambogyl (4-(2-pyridyl)piperazine); 10-(4-(2-pyridyl)piperazinyl)gambogyl (4-(2-pyridyl)piperazine); 6-acetylgambogic acid; 10-(4-(2-pyridyl)piperazinyl)gambogic acid; N-hydroxy-succinimidyl gambogate; 8-(gambogylamido)octanoic acid; 6-(gambogylamido)hexanoic acid; 12-(gambogylamido)dodecanoic acid; N-hydroxysuccinimidyl-8-(gambogylamido)-octanoate; N-hydroxysuccinimidyl-6-(gambogylamido)hexanoate; N-hydroxy-succinimidyl-12-(gambogylamido)dodecanoate; 10-methoxy-gambogyl piperidine; gambogyl (4-(2-pyrimidyl)piperazine); gambogyl (bis(2-pyridylmethyl)amine); gambogyl (N-(3-pyridyl)-N-(2-hydroxybenzyl)amine); gambogyl (4-benzylpiperazine); gambogyl (4-(3,4-methylenedioxybenzyl)piperazine); gambogyl (N-methyl-5-(methylamino)-3-oxapentylamine); gambogyl (N-methyl-8-(methylamino)-3,6-dioxaoctylamine); gambogyl (N-ethyl-2-(ethylamino)ethylamine); Gambogyl (4-isopropylpiperazine); gambogyl (4-cyclopentylpiperazine); gambogyl (N-(2-oxo-2-ethoxyethyl)-(2-pyridyl)methylamine); gambogyl (2,5-dimethylpiperazine); gambogyl (3,5-dimethylpiperazine); gambogyl (4-(4-acetylphenyl)piperazine); gambogyl (4-ethoxycarbonylpiperazine); gambogyl (4-(2-oxo-2-pyrrolidylethyl)piperazine); gambogyl (4-(2-hydroxyethyl)piperazine); gambogyl (N-methyl-2-(methylamino)ethylamine); gambogyl (N-methyl-2-(benzylamino)ethylamine); gambogyl (N-methyl-(6-methyl-2-pyridyl)methylamine); gambogyl (N-ethyl-2-(2-pyridyl)ethylamine); gambogyl (N-methyl-(2-pyridyl)methylamine); gambogyl (N-methyl-4-(3-pyridyl)butylamine); gambogyl (bis(3-pyridylmethyl)amine); gambogyl (2,4-dimethyl-2-imidazoline); gambogyl (4-methyl-homopiperazine); gambogyl (4-(5-hydroxy-3-oxapentyl)piperazine); gambogyl (3-dimethylaminopyrrolidine); gambogyl ((2-furanyl)methylamine); gambogyl (2-hydroxy-1-methyl-2-phenylethylamine); gambogyl (3,4,5-trimethoxybenzylamine); gambogyl (2-(2-methoxyphenyl)ethylamine); gambogyl (2-methoxybenzylamine); gambogyl (3,4-methylenedioxybenzylamine); gambogyl (2-(2,5-dimethoxyphenyl)-ethylamine); gambogyl (2-(3-methoxyphenyl)ethylamine); gambogyl (3-(piperidinyl)propylamine); gambogyl (2-(piperidinyl)ethylamine); gambogyl (3,4-dimethoxybenzylamine); gambogyl ((2-tetrahydrofuranyl)methylamine); gambogyl ((N-ethyl-2-pyrrolidinyl)methylamine); gambogyl (2-diethylaminoethylamine); gambogyl (2,2-dimethyl-3-dimethylaminopropylamine); gambogyl ((N-ethoxycarbonyl-4-piperidinyl)amine); gambogyl (2-carbamylpyrrolidine); gambogyl (3-(homopiperidinyl)propylamine); gambogyl ((N-benzyl-4-piperidinyl)amine); gambogyl (2-(4-methoxyphenyl)ethylamine); gambogyl (4-oxa-hex-5-enylamine); gambogyl (6-hydroxyhexylamine); gambogyl (2-(3,5-dimethoxyphenyl)ethylamine); gambogyl (3,5-dimethoxybenzylamine); and gambogyl (2-carbamyl-2-(4-hydroxyphenyl)ethylamine). In still further embodiments, said subject is a mammal. In additional embodiments, said composition is a solution. In some embodiments, the mode of said administration is selected from the group consisting of optical, oral, parenteral, mucosol, buccal, vaginal, rectal, sublingual, inhalation, insufflation, intravenous, intrathecal, subcutaneous and intramuscular. In further embodiments, said disease is selected from the group consisting of cornea angiogenesis, diabetic retinopathy, inflammation, rheumatoid arthritis, psoriasis and impaired wound healing.

In some embodiments, the invention relates to a method of treating macular degeneration in a mammal comprising: providing: a mammal exhibiting symptoms associated with macular degeneration, and a composition comprising gambogic acid or a gambogic acid derivative; and administering said composition to said mammal under conditions such that said symptoms are reduced. In further embodiments, said gambogic acid derivative is selected from the group consisting of methyl gambogate; 9,10-dihydrogambogic acid; 9,10-dihydrogambogyl (4-methylpiperazine); 9,10-dihydrogambogyl (2-dimethylaminoethylamine); gambogyl diethylamine; gambogyl dimethylamine; gambogyl amine; gambogyl hydroxyamine; gambogyl piperidine; 6-methoxy-gambogic acid; 6-(2-dimethylaminoethoxy)-gambogic acid; 6-(2-piperidinylethoxy)-gambogic acid; 6-(2-morpholinylethoxy)-gambogic acid; 6-methoxy-gambogyl piperidine; gambogyl morpholine; gambogyl (2-dimethylaminoethylamine); 10-morpholinyl-gambogyl morpholine; 10-morpholinyl-gambogyl piperidine; 10-(4-methylpiperazinyl)-gambogyl piperidine; 10-(4-methylpiperazinyl)-gambogyl morpholine; 10-piperidinyl-gambogyl piperidine; 10-(4-methylpiperazinyl)-gambogyl (4-methylpiperazine); gambogyl (4-methylpiperazine); methyl-6-methoxy-gambogate; gambogenic acid; gambogenin; 10-methoxy-gambogic acid; 10-butylthio-gambogic acid; 10-(4-methylpiperazinyl)-gambogic acid; 10-pyrrolidinyl-gambogic acid; methyl-10-morpholinyl-gambogate; 10-piperidinyl-gambogic acid; 10-morpholinyl-gambogic acid; N-(2-gambogylamidoethyl)biotinamide; gambogyl (2-morpholinylethylamine); 9,10-epoxygambogic acid; gambogyl (4-(2-pyridyl)piperazine); 10-(4-(2-pyridyl)-piperazinyl)gambogyl (4-(2-pyridyl)piperazine); 6-acetylgambogic acid; 10-(4-(2-pyridyl)piperazinyl)gambogic acid; N-hydroxysuccinimidyl gambogate; 8-(gambogylamido)octanoic acid; 6-(gambogylamido)hexanoic acid; 12-(gambogylamido)-dodecanoic acid; N-hydroxysuccinimidyl-8-(gambogylamido)octanoate; N-hydroxy-succinimidyl-6-(gambogylamido)hexanoate; N-hydroxysuccinimidyl-12-(gambogyl-amido)dodecanoate; 10-methoxy-gambogyl piperidine; gambogyl (4-(2-pyrimidyl)piperazine); gambogyl (bis(2-pyridylmethyl)amine); gambogyl (N-(3-pyridyl)-N-(2-hydroxybenzyl)amine); gambogyl (4-benzylpiperazine); gambogyl (4-(3,4-methylenedioxybenzyl)piperazine); gambogyl (N-methyl-5-(methylamino)-3-oxapentylamine); gambogyl (N-methyl-8-(methylamino)-3,6-dioxaoctylamine); gambogyl (N-ethyl-2-(ethylamino)ethylamine); Gambogyl (4-isopropylpiperazine); gambogyl (4-cyclopentylpiperazine); gambogyl (N-(2-oxo-2-ethoxyethyl)-(2-pyridyl)methylamine); gambogyl (2,5-dimethylpiperazine); gambogyl (3,5-dimethylpiperazine); gambogyl (4-(4-acetylphenyl)piperazine); gambogyl (4-ethoxycarbonylpiperazine); gambogyl (4-(2-oxo-2-pyrrolidylethyl)piperazine); gambogyl (4-(2-hydroxyethyl)piperazine); gambogyl (N-methyl-2-(methylamino)ethylamine); gambogyl (N-methyl-2-(benzylamino)ethylamine); gambogyl (N-methyl-(6-methyl-2-pyridyl)methylamine); gambogyl (N-ethyl-2-(2-pyridyl)ethylamine); gambogyl (N-methyl-(2-pyridyl)methylamine); gambogyl (N-methyl-4-(3-pyridyl)butylamine); gambogyl (bis(3-pyridylmethyl)amine); gambogyl (2,4-dimethyl-2-imidazoline); gambogyl (4-methyl-homopiperazine); gambogyl (4-(5-hydroxy-3-oxapentyl)piperazine); gambogyl (3-dimethylaminopyrrolidine); gambogyl ((2-furanyl)methylamine); gambogyl (2-hydroxy-1-methyl-2-phenylethylamine); gambogyl (3,4,5-trimethoxybenzylamine); gambogyl (2-(2-methoxyphenyl)ethylamine); gambogyl (2-methoxybenzylamine); gambogyl (3,4-methylenedioxybenzylamine); gambogyl (2-(2,5-dimethoxyphenyl)-ethylamine); gambogyl (2-(3-methoxyphenyl)ethylamine); gambogyl (3-(piperidinyl)propylamine); gambogyl (2-(piperidinyl)ethylamine); gambogyl (3,4-dimethoxybenzylamine); gambogyl ((2-tetrahydrofuranyl)methylamine); gambogyl ((N-ethyl-2-pyrrolidinyl)methylamine); gambogyl (2-diethylaminoethylamine); gambogyl (2,2-dimethyl-3-dimethylaminopropylamine); gambogyl ((N-ethoxycarbonyl-4-piperidinyl)amine); gambogyl (2-carbainylpyrrolidine); gambogyl (3-(homopiperidinyl)propylamine); gambogyl ((N-benzyl-4-piperidinyl)amine); gambogyl (2-(4-methoxyphenyl)ethylamine); gambogyl (4-oxa-hex-5-enylamine); gambogyl (6-hydroxyhexylamine); gambogyl (2-(3,5-dimethoxyphenyl)ethylamine); gambogyl (3,5-dimethoxybenzylamine); and gambogyl (2-carbamyl-2-(4-hydroxyphenyl)ethylamine). In still further embodiments, said composition is a solution. In additional embodiments, the mode of said administration is selected from the group consisting of optical, oral, parenteral, mucosol, buccal, vaginal, rectal, sublingual, inhalation, insufflation, intravenous, intrathecal, subcutaneous and intramuscular.

In some embodiments, the invention relates to a method for treating a disease characterized by angiogenesis comprising: providing: a mammal exhibiting symptoms associated with said disease, and a composition comprising gambogic acid (GA) or a gambogic acid derivative; and administering said composition to said mammal under conditions such that said symptoms are reduced. In further embodiments, said gambogic acid derivative is selected from the group set forth above. In still further embodiments, said composition is a solution. In additional embodiments, the mode of said administration is selected from the group consisting of optical, oral, parenteral, mucosol, buccal, vaginal, rectal, sublingual, inhalation, insufflation, intravenous, intrathecal, subcutaneous and intramuscular. In some embodiments, said disease is selected from the group consisting of cornea angiogenesis, diabetic retinopathy, inflammation, rheumatoid arthritis, psoriasis and impaired wound healing. We find GA significantly inhibits corneal angiogenesis in a mouse corneal model (FIG. 8). With the mouse oxygen-induced ischemic retinopathy (OIR) model, we identify that GA can significantly inhibits retinal neovascularization (FIG. 9). Therefore, the present invention contemplates GA (and derivatives thereof) as a potent drug for diabetic retinopathy, age-related macular degeneration (AND), and retinopathy of prematurity (ROP).

### BRIEF DESCRIPTION OF THE DRAWINGS

For a more complete understanding of the features and advantages of the present invention, reference is now made to the detailed description of the invention along with the accompanying figures.

FIG. 1 shows the general chemical structure of gambogic acid (GA) (FIG. 1A) and further demonstrates its apoptotic activity against cancer cells. Whole cell proteins of GA treated human vascular endothelial cells (HUVECS) were analyzed by Western blotting with anti-cleaved caspase 3 and anti-cleaved PARP antibodies The cleaved caspase 3 (FIG. 1B) and PARP (FIG. 1C) shows that GA induces apoptosis at 100 nM in HUVEC cells. Proliferation assays performed on HUVEC cells using differing concentrations of GA were performed and compared to the effects on prostate cancer 3 (PC3) cells. 80 nM GA was found to inhibit HUVEC cell proliferation by 50% (FIG. 1D) while more than 400 nM GA is required to obtain the same effect in PC3 cancer cells (FIG. 1E).

FIG. 2 shows GA suppression of VEGF induced cell migration. 10 nM GA inhibits VEGF dependent migration of HUVEC cells (FIGS. 2A and 2B). More than 100 nM GA was required to inhibit VEGF migration for PC3 cancer cells (FIG. 2C).

FIG. 3 shows GA inhibition of HUVEC cell invasion and tube formation. The effect of GA on endothelial cell invasion was evaluated by performing transwell assays. 40 nM GA was found to inhibit almost all invasion activities of HUVEC cells (FIG. 3A). Tube formation by endothelial cells was evaluated using HUVEC cells (4×10⁴ cells) in 1 ml endothelial cell growth medium (ECGM) with differing concentrations of GA on Matrigel layers. Approximately 50 mM GA inhibited 50% tube formation of HUVEC cells on Matrigel assays while 100 nM GA completely inhibited the tube formation ability of HUVECs on Matrigel (FIG. 3B).

FIG. 4 shows that GA inhibits angiogenesis in vitro and in vivo. FIG. 4A shows that GA inhibits angiogenesis in vitro. About 1-1.5 mm long cleaned mice aortic rings were placed on the Matrigel covered wells and covered with another 100 μl of Matrigel. After 4 days incubation with 1.5 ml of ECGM medium with or without GA, images were taken with Olympus IX 70 invert microscope and vessels were counted. FIG. 4B shows that GA inhibits angiogenesis in vivo. Matrigel (0.5 ml/plug) with neither GA nor VEGF, VEGF (4 ng/ml) but no GA, VEGF (4 ng/ml) and 0.1 μM GA, VEGF (4 ng/ml) and 0.2 μM GA were injected subcutaneously in the midventral abdominal region of 5-6 week old C57BL/6 mice (five mice for each group). After 7 days, the mice were sacrificed and the plugs were removed. The Matrigel plugs were fixed with formalin and embedded with paraffin and the 5 μm sections were stained with H&E staining. The angiogenesis inhibition effect of GA in outer vessel layer of Matrigel plugs is shown. The vessels in Matrigel plugs were counted and the angiogenesis inhibition effect of GA in vivo is shown.

FIG. 5 shows that GA inhibits tumor-angiogenesis and prevents tumor growth in vivo. Log-phase PC3 human prostate tumor cells were injected s.c. (2×10⁶ cell per mouse) into the 5-6 week old SCID male mice right flank. After the tumors had become established (about 50 mm³), the mice were injected with or without 3 mg/kg GA every day. After 15 days, mice were sacrificed and tumors were removed and taken images by Nikon camera. FIG. 5A is a graph that shows that tumors of the control group increased from 51.18±5.3 mm³ to 1144±169 mm³, while that from GA treated group increased only from 51.74±3.8 mm³ to 127.4±25.6 mm³. FIG. 5B shows that tumors from the mice with GA treatment were significantly smaller than that from control group. FIG. 5C is a graph that shows that tumors from the control group were 0.28±0.08 g while from groups treated with GA were 0.012±0.0008 g in average. FIG. 5D shows tumors that were fixed with Histochoice® MB (Molecular Biology) tissue fixative (Amresco®) and embedded with paraffin. The 5 μm sections were performed blood vessel staining. FIG. 5E is a graph that shows that the average vessel number in tumors of the control group was 14±2 per high performance field (HPF, 200×) while that in GA treated group was 1.8±1.3 per HPF. FIG. 5F is a graph that shows that the average body weight of control group mice decreased from 22.3±1.2 g to 21.2±1.3 g, while that of the GA treated group increased from 22.4±1 g to 24.6±0.9 g.

FIG. 6 shows that GA inhibits VEGF receptor 2 (VEGFR2) kinase activation and VEGFR2 downstream signals. FIG. 6A shows that 1 nM of GA strongly inhibited VEGFR2 kinase activity. After starvation in ECGM medium without serum overnight, HUVEC cells were washed with 1×PBS twice, followed with incubation in M199 medium. HUVEC cells were then treated with 1 nM of GA and/or 4 nM of VEGF for 5 minutes, then subjected to immunoprecipitation using anti-VEGFR2 antibody. Anti-phosphotyrosine antibody was used for the detection of phosphorylation of the tyrosine residue of VEGFR2. FIG. 6B is a graph that shows that GA inhibits VEGFR2 activation. Reaction cocktail containing 100 ng of active VEGFR2 was incubated with different concentrations of GA for 5 minutes at room temperature. Substrate peptide cocktail was added to the pre-incubated reaction cocktail/GA compound. After incubation at room temperature for 30 minutes, stop buffer was added to halt the reaction. 25 μl of each reaction was then transferred with 75 μl dH2O per well into a 96-well streptavidin coated plate and incubated at room temperature for 60 minutes. After washing three times with 200 μl/well PBS/T, a 100 μl primary antibody (Phosphotyrosine Monoclonal Antibody (P-Tyr-100)) was added. Incubation at room temperature for 60 minutes was followed with PBS washing. A 100 μl diluted HRP labeled anti-mouse IgG was then added. Following incubation at room temperature for 30 minutes, the wells were washed five times with 200 μl PBS/T. A 100 μl per well TMB substrate was then added and incubated for 15 minutes. The stop solution was added and the plate was detected at 405 nm. IC₅₀=12 pM. The IC₅₀ was identified as the concentration of GA to inhibit 50% of the activity of 100 ng VEGFR2. FIG. 6C shows that GA suppressed VEGFR2 downstream signal activation with or without VEGF induction via c-Src, FAK and AKT. HUVEC cells pretreated with or without 4 nM VEGF for 5 minutes were treated with GA (0, 1, 5, 10, 20 and 40 nM) for an additional 5 minutes. Whole cell proteins (200 μg) of each sample were isolated and immunoprecipitated with anti-c-Src, FAK and AKT antibodies and Western blotting with pTyr-antibody for c-Src phosphorylation, pFAK397 antibody for c-Src-associated FAK phosphorylation at multiple tyrosine residues and pSer473-AKT antibody for AKT phosphorylation. The phosphorylation inhibition effect of GA on c-Src, FAK, AKT is shown. FIG. 6D shows a diagram of the GA directed anti-angiogenesis mechanism.

FIG. 7 shows the general chemical structure for gambogic acid and some of its derivatives. The table provides for some of the potential chemical substituents. It is not intended that the present invention be limited to the chemical species and substituents described in FIG. 7.

Table 1 shows that GA, at a concentration of 80 nM, induces apoptosis in only 4% of PC3 cells while in HUVECs GA induced apoptosis in 40% of the cells. This data suggests that GA was more effective in promoting apoptosis in endothelial cells as compared to cancer cells.

FIG. 8 shows that GA inhibits VEGF induced corneal angiogenesis. FIG. 8A shows four pictures of mouse eyes: control (top left), 160 ng/ml VEGF (top right) which causes extensive angiogenesis, 2.5 ug/ml GA (bottom left), and 160 ng/ml+2.5 ug/ml GA (bottom right) which shows that 2.5 μg/ml GA dramatically inhibits 160 ng/ml VEGF induced angiogenesis in mouse cornea. FIG. 8B graphically shows that GA inhibits VEGF induced corneal angiogenesis [the statistic data are calculated from three independent experiments (P<0.05)].

FIG. 9 shows that GA inhibits hypoxia-induced retina angiogenesis. FIG. 9A shows wholemount fluorescein-dextran staining of retinal vasculature from P17 normal (A1, 21% oxygen), Hypoxia (A2, 75%), enlarged area (see box) from A2 (A3), and GA-treated mice exposed to OIR (A4), respectively. FIG. 9B is a bar graph providing a quantitative assessments of retinal neovascularization in eyes from P17 control and GA-treated mice exposed to OIR. Data in each column are the mean SD values from four eyes of four mice. Note that there is a significant difference in the degree of neovascularization among control and GA-treated mice (***□ p<0.001). GA concentration was used as 10 mg/kg for this mouse model. Assessment of retinal neovasculature in control (left) and GA-treated (right) mice during OIR.P7 mice were exposed to a cycle of hyperoxia and normoxia, and eyes were removed for appropriate analysis. The abnormal angiogenesis associated with vitreal invasion can be seen as glomeruloid-like tufts of cell (arrow).

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “gambogic acid” refers to a compound represented by the following chemical structure:

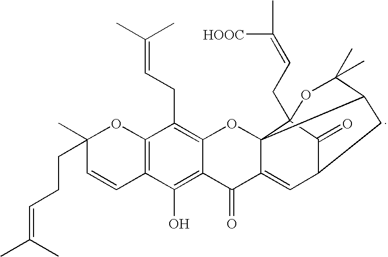

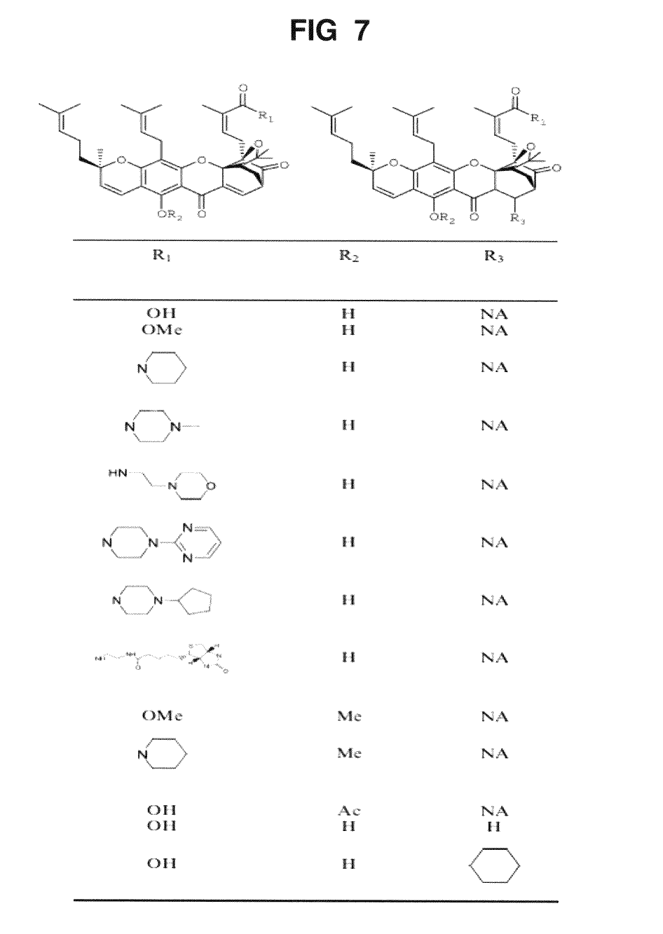

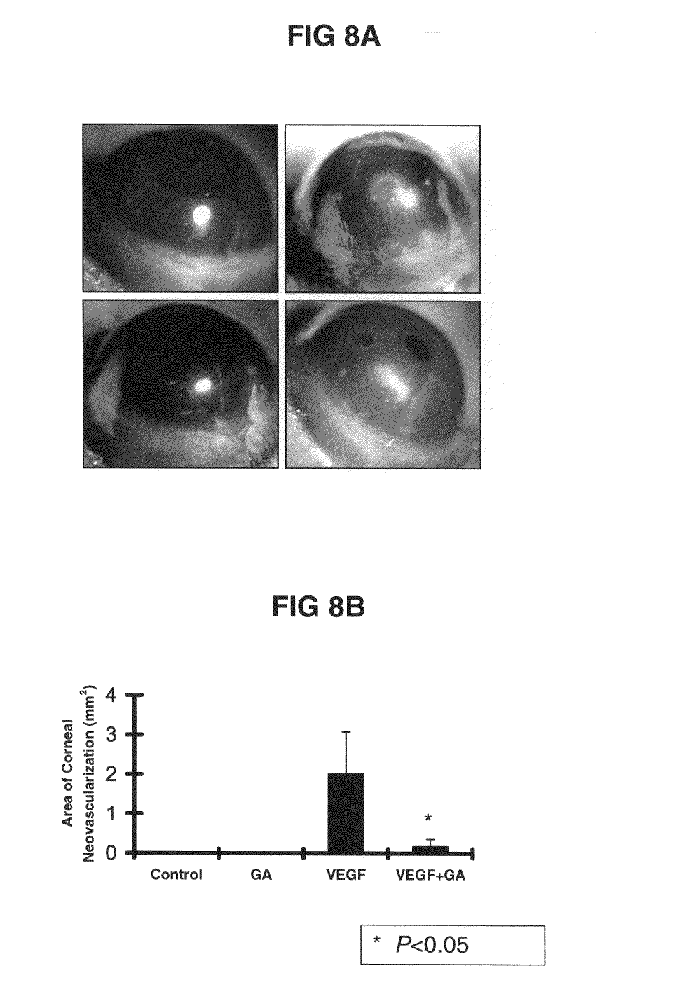

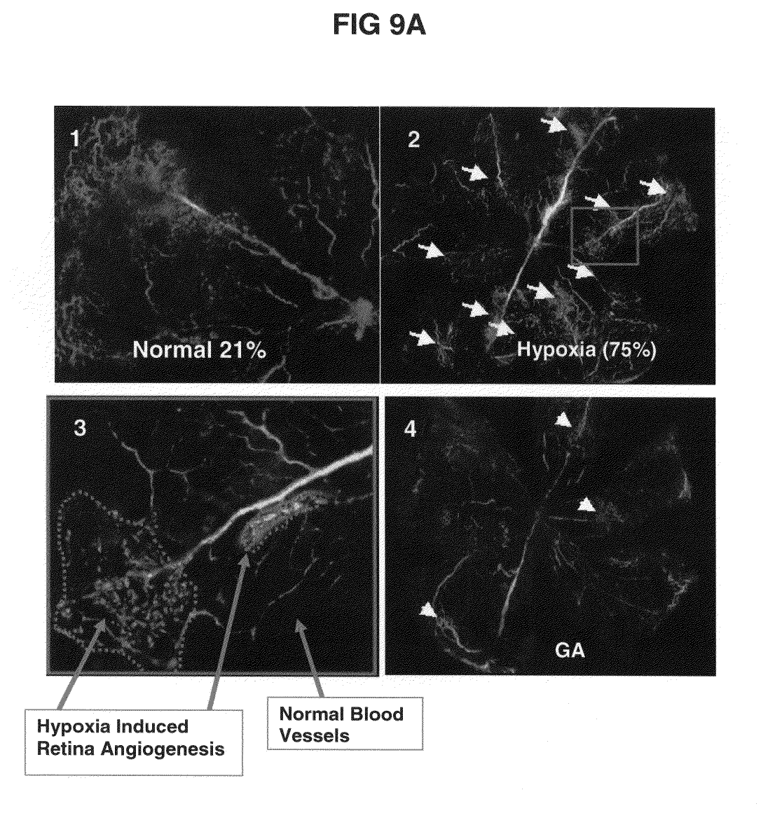

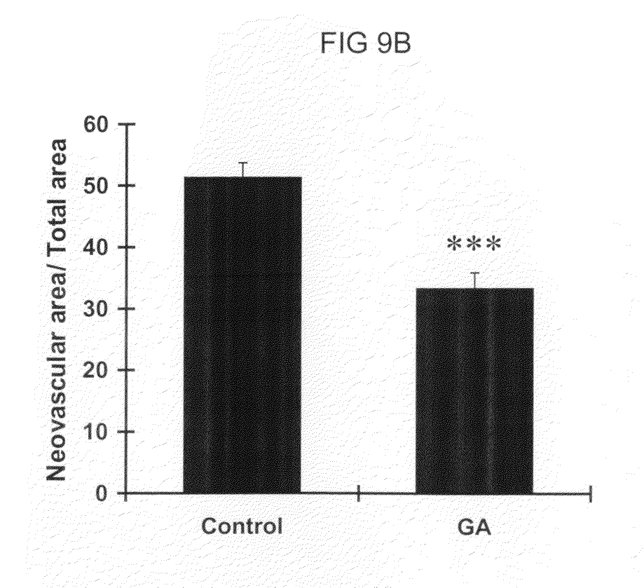

It is not intended that the invention be limited to any particular derivative, analog or isomer of gambogic acid. Examples of derivatives of gambogic acid include but are in no way limited to methyl gambogate; 9,10-dihydrogambogic acid; 9,10-dihydrogambogyl (4-methylpiperazine); 9,10-dihydrogambogyl (2-dimethylaminoethylamine); gambogyl diethylamine; gambogyl dimethylamine; gambogyl amine; gambogyl hydroxyamine; gambogyl piperidine; 6-methoxy-gambogic acid; 6-(2-dimethylaminoethoxy)-gambogic acid; 6-(2-piperidinylethoxy)-gambogic acid; 6-(2-morpholinylethoxy)-gambogic acid; 6-methoxy-gambogyl piperidine; gambogyl morpholine; gambogyl (2-dimethylaminoethylamine); 10-morpholinyl-gambogyl morpholine; 10-morpholinyl-gambogyl piperidine; 10-(4-methylpiperazinyl)-gambogyl piperidine; 10-(4-methylpiperazinyl)-gambogyl morpholine; 10-piperidinyl-gambogyl piperidine; 10-(4-methylpiperazinyl)-gambogyl (4-methylpiperazine); gambogyl (4-methylpiperazine); methyl-6-methoxy-gambogate; gambogenic acid; gambogenin; 10-methoxy-gambogic acid; 10-butylthio-gambogic acid; 10-(4-methylpiperazinyl)-gambogic acid; 10-pyrrolidinyl-gambogic acid; methyl-10-morpholinyl-gambogate; 10-piperidinyl-gambogic acid; 10-morpholinyl-gambogic acid; N-(2-gambogylamidoethyl)biotinamide; gambogyl (2-morpholinylethylamine); 9,10-epoxygambogic acid; gambogyl (4-(2-pyridyl)piperazine); 10-(4-(2-pyridyl)piperazinyl)gambogyl (4-(2-pyridyl)piperazine); 6-acetylgambogic acid; 10-(4-(2-pyridyl)piperazinyl)gambogic acid; N-hydroxy-succinimidyl gambogate; 8-(gambogylamido)octanoic acid; 6-(gambogylamido)hexanoic acid; 12-(gambogylamido)dodecanoic acid; N-hydroxysuccinimidyl-8-(gambogylamido)-octanoate; N-hydroxysuccinimidyl-6-(gambogylamido)hexanoate; N-hydroxy-succinimidyl-12-(gambogylamido)dodecanoate; 10-methoxy-gambogyl piperidine; gambogyl (4-(2-pyrimidyl)piperazine); gambogyl (bis(2-pyridylmethyl)amine); gambogyl (N-(3-pyridyl)-N-(2-hydroxybenzyl)amine); gambogyl (4-benzylpiperazine); gambogyl (4-(3,4-methylenedioxybenzyl)piperazine); gambogyl (N-methyl-5-(methylamino)-3-oxapentylamine); gambogyl (N-methyl-8-(methylamino)-3,6-dioxaoctylamine); gambogyl (N-ethyl-2-(ethylamino)ethylamine); Gambogyl (4-isopropylpiperazine); gambogyl (4-cyclopentylpiperazine); gambogyl (N-(2-oxo-2-ethoxyethyl)-(2-pyridyl)methylamine); gambogyl (2,5-dimethylpiperazine); gambogyl (3,5-dimethylpiperazine); gambogyl (4-(4-acetylphenyl)piperazine); gambogyl (4-ethoxycarbonylpiperazine); gambogyl (4-(2-oxo-2-pyrrolidylethyl)piperazine); gambogyl (4-(2-hydroxyethyl)piperazine); gambogyl (N-methyl-2-(methylamino)ethylamine); gambogyl (N-methyl-2-(benzylamino)ethylamine); gambogyl (N-methyl-(6-methyl-2-pyridyl)methylamine); gambogyl (N-ethyl-2-(2-pyridyl)ethylamine); gambogyl (N-methyl-(2-pyridyl)methylamine); gambogyl (N-methyl-4-(3-pyridyl)butylamine); gambogyl (bis(3-pyridylmethyl)amine); gambogyl (2,4-dimethyl-2-imidazoline); gambogyl (4-methyl-homopiperazine); gambogyl (4-(5-hydroxy-3-oxapentyl)piperazine); gambogyl (3-dimethylaminopyrrolidine); gambogyl ((2-furanyl)methylamine); gambogyl (2-hydroxy-1-methyl-2-phenylethylamine); gambogyl (3,4,5-trimethoxybenzylamine); gambogyl (2-(2-methoxyphenyl)ethylamine); gambogyl (2-methoxybenzylamine); gambogyl (3,4-methylenedioxybenzylamine); gambogyl (2-(2,5-dimethoxyphenyl)ethylamine); gambogyl (2-(3-methoxyphenyl)ethylamine); gambogyl (3-(piperidinyl)propylamine); gambogyl (2-(piperidinyl)ethylamine); gambogyl (3,4-dimethoxybenzylamine); gambogyl ((2-tetrahydrofuranyl)methylamine); gambogyl ((N-ethyl-2-pyrrolidinyl)methylamine); gambogyl (2-diethylaminoethylamine); gambogyl (2,2-dimethyl-3-dimethylaminopropylamine); gambogyl ((N-ethoxycarbonyl-4-piperidinyl)amine); gambogyl (2-carbamylpyrrolidine); gambogyl (3-(homopiperidinyl)propylamine); gambogyl ((N-benzyl-4-piperidinyl)amine); gambogyl (2-(4-methoxyphenyl)ethylamine); gambogyl (4-oxa-hex-5-enylamine); gambogyl (6-hydroxyhexylamine); gambogyl (2-(3,5-dimethoxyphenyl)ethylamine); gambogyl (3,5-dimethoxybenzylamine); and gambogyl (2-carbamyl-2-(4-hydroxyphenyl)ethylamine). While in no way limiting the scope of the present invention, some of the derivatives are provided in FIG. 7. Derivatives of gambogic acid may be synthesized using methods known to those skilled in the art as well as those described in Cai et al., United States Patent Application Number 20070093456, and Zhang et al., Bioorganic and Medicinal Chemistry 12, 309-317 (2004), both of which are hereby incorporated by reference. It is not intended that the present invention be limited by the type of chemical substituent or substituents that is or are coordinated to gambogic acid. Examples of chemical substituents include but are in no way limited to hydrogen, methyl, ethyl, formyl, acetyl, phenyl, chloride, bromide, hydroxyl, methoxyl, ethoxyl, methylthiol, ethylthiol, propionyl, carboxyl, methoxy carbonyl, ethoxycarbonyl, methylthiocarbonyl, ethylthiocarbonyl, butylthiocarbonyl, dimethylcarbamyl, diethylcarbamyl, N-piperidinylcarbonyl, N-methyl-N′-piperazinylcarbonyl, 2-(dimethylamino)ethylcarboxy, N-morpholinylcarbonyl, 2-(dimethylamino)ethylcarbamyl, 1-piperidinylcarbonyl, methylsulfonyl, ethylsulfonyl, phenylsulfonyl, 2-piperidinylethyl, 2-morpholinylethyl, 2-(dimethylamino)ethyl, 2-(diethylamino)ethyl, butylthiol, dimethylamino, diethylamino, piperidinyl, pyrrolidinyl, imidazolyl, pyrazolyl, N-methylpiperazinyl, 2-(dimethylamino)ethylamino or morpholinyl.

As used herein, “angiogenesis” refers to a physiological process involving the growth of new blood vessels from pre-existing vessels. Vasculogenesis is the term used for spontaneous blood-vessel formation, and intussusception is the term for new blood vessel formation by splitting off existing ones. Angiogenesis is a normal process in growth and development, as well as in wound healing. However, this is also a fundamental step in the transition of tumors from a dormant state to a malignant state. VEGF (Vascular Endothelial Growth Factor) has been demonstrated to be a major contributor to angiogenesis, increasing the number of capillaries in a given network. Upregulation of VEGF is a major component of the physiological response to exercise and its role in angiogenesis is suspected to be a possible treatment in vascular injuries. In vitro studies clearly demonstrate that VEGF is a potent stimulator of angiogenesis because, in the presence of this growth factor, plated endothelial cells will proliferate and migrate, eventually forming tubular structures resembling capillaries. VEGF causes a massive signaling cascade in endothelial cells. While the present invention is not limited to any particular mechanism, it is believed that binding to VEGF receptor 2 (VEGFR2) starts a tyrosine kinase signaling cascade that stimulates the production of factors that variously stimulate vessel permeability (eNOS, producing NO), proliferation/survival (bFGF), migration (ICAMs/VCAMs/MMPs) and finally differentiation into mature blood vessels. Mechanically, VEGF is upregulated with muscle contractions as a result of increased blood flow to affected areas. The increased flow also causes a large increase in the mRNA production of VEGF receptors 1 and 2.

As used herein, “macular degeneration” means any condition that causes part of the macula to deteriorate. This degeneration may be partial or total, and it is not intended to be limited to advance stages of the disease. A symptom of macular degeneration is a change in central vision. The patient may notice blurred central vision or a blank spot on the page when reading. The patient may notice visual distortion such as bending of straight lines. Images may appear smaller. Some patients notice a change in color perception and some experience abnormal light sensations. These symptoms may come on suddenly and become progressively more troublesome. Sudden onset of symptoms, particularly vision distortion, is an indication for immediate evaluation by an ophthalmologist. Examples of symptoms associated with macular degeneration include but are not limited to diminished or changes color perception, the appearance of dark, blurry areas or white out in the center of vision, the distortion of straight lines and the distortion of the center of vision.

“Diabetes” or “diabetes mellitus” refers to a syndrome characterized by disordered metabolism and inappropriately high blood sugar (hyperglycemia) resulting from either low levels of the hormone insulin or from abnormal resistance to insulin's effects coupled with inadequate levels of insulin secretion to compensate. A diabetic (Type I or Type II) patient is at risk for macular degeneration. Diabetic macular degeneration is the deterioration of the macula due to diabetes. Examples of symptoms associated with diabetes include but are not limited to increased thirst and appetite, dry mouth, frequent urination, fatigue, blurred vision, headaches and unexplained weight loss. Pathological angiogenesis in the retina is the leading cause of human blindness resulting from diabetic retinopathy, age-related macular degeneration (AMD), and retinopathy of prematurity (ROP). In the most severe form of age-related macular degeneration, known as “wet” AMD, abnormal angiogenesis occurs under the retina resulting in irreversible loss of vision. The loss of vision is due to scarring of the retina secondary to the bleeding from the new blood vessels. Approximately 10% of patients with age-related macular degeneration will grow abnormal blood vessels under their retinas and thus progress from the “dry” form to the “wet” form of AMD.

As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced. We have identified Gambogic acid (GA) as (in one embodiment) an inhibitor of angiogenesis (neovascularization) by targeting vascular endothelial growth factor receptor 2 (VEGFR2) and its downstream signaling pathways. Furthermore, GA inhibits angiogenesis in both cornea and retina as shown in the data (below), suggesting GA could be used as a novel agent targeting retina angiogenesis in “wet” AMD.

“Subject” refers to any mammal, preferably a human patient, livestock, or domestic pet.

In a specific embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

### DETAILED DESCRIPTION OF THE INVENTION

The present invention relates to methods and compositions for the treatment of angiogenesis and macular degeneration. In preferred embodiments, the invention relates to the field of eye health. In some embodiments, the invention relates to the prevention and treatment of angiogenesis by administering compounds disclosed herein. In further embodiments, the invention relates to the prevention and treatment of macular degeneration by administering compounds disclosed herein. In still further embodiments, the invention relates to methods and compositions comprising gambogic acid and gambogic acid derivatives.

Angiogenesis is crucial for organ growth and repair; however, an imbalance in this process contributes to numerous diseases. Excessive angiogenesis leads to inflammation, rheumatoid arthritis, psoriasis, diabetic retinopathy, impaired wound healing, and cancer. Angiogenesis is also a key step in cancer growth and metastasis. Thus, inhibiting angiogenesis is a promising strategy for the treatment of cancer and other diseases and therapeutic angiogenesis is an exciting frontier of cancer and cardiovascular medicine.

Vascular endothelial growth factors (VEGF) and VEGF receptor signals represent a critical rate-limiting step in physiological angiogenesis. VEGFs exert their effects after binding in an overlapping pattern to three receptor tyrosine kinases known as VEGF receptor-1, -2 and -3 (VEGFR1-3), as well as to co-receptors such as heparin sulfate proteoglycans and neuropilins. VEGF receptors undergo ligand-induced homodimerization or heterodimerization, which activates their intrinsic tyrosine kinase activity. VEGFR1 is poorly autophosphorylated in response to VEGF in endothelial cells and is weakly involved in transducing the VEGF angiogenic signals. VEGFR3 mainly functions in the establishment and maintenance of the lymphatic system. In contrast, ligand-induced homodimerization of VEGFR2 leads to a strong autophosphorylation of VEGFR2 on tyrosine residues, which drives the activation of major VEGF signaling pathways. Major autophosphorylation sites on VEGFR2 have been described as Y1175, Y951, Y1214, Y1054 and Y10595. In particular, phosphorylation of Y1175 by VEGF is crucial to initiate the activation of phospholipase C-γ (PLC-γ) that mediates signal-to-cell proliferation and vascular permeability. Phosphorylation of Tyr-1175 is also required for binding and activation of Shb and phosphoinositide-3 kinase (PI3K), which is critical for subsequent activation of endothelial cell survival, migration and proliferation. Phosphorylation of Y951 is necessary for binding and activation of T cell-specific adapter protein (TSAD) and Src, which regulates cell migration and vascular permeability. Phosphorylation of Tyr-1214 of VEGFR2 is necessary for the activation of stress-activated protein kinase 2/p38 and its direct target MAPK, which regulates actin remodeling and cell migration through mediating the activation of Heat-shock-protein-27 (HSP27). It is well known that VEGFR2 is the primary receptor mediating the angiogenic activity of VEGF through distinct signal transduction pathways that regulate endothelial cell proliferation, migration, differentiation, and tube formation.

In some embodiments, the invention relates to methods and compositions comprising gambogic acid. Gambogic acid (GA, C₃₈H₄₄O₈; MW 628.76), a polyprenylated xanthone, is the main active compound of Gamboge Hanburyi (a traditional Chinese medicine) used for detoxification, homeostasis and as a pesticide for centuries. Previous studies showed that GA exhibited a variety of effects by activating apoptosis in the human gastric cancer line BGC-823, the human gastric carcinoma MGC-803 cells, and T47D breast cancer cells; by inhibiting proliferation in human hepatoma SMMC-7721 and lung carcinoma SPC-A1 cells; and by arresting G2/M cell cycle in human gastric carcinoma BGC-823 cells. Recent reports demonstrated that GA triggered apoptosis and prevented cancer cell proliferation by binding to transferring receptor and by suppressing nuclear factor kappa B (NF-kappaB) signaling pathway.

Angiogenesis is crucial for cancer progression and metastasis. The current invention examines GA's ability to inhibit angiogenesis in vitro and in vivo. GA was found to inhibit angiogenesis. GA was further identified as a novel VEGFR2 inhibitor. GA further inhibited HUVEC cell proliferation, migration, tube formation, microvessel growth, and angiogenesis using in vitro and in vivo approaches. Using a xenograft model, GA was found to inhibit tumor angiogenesis and tumor growth by blocking angiogenesis. The inhibitory effects of GA on angiogenesis are mediated through suppressing VEGFR2 and its downstream signaling pathways. Thus, GA is a new drug candidate in anti-angiogenesis and anti-cancer therapies.

### Pharmaceutical Formulations

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155).

In a preferred embodiment, the active compound and optionally another therapeutic or prophylactic agent are formulated in accordance with routine procedures as pharmaceutical compositions adapted for intravenous administration to human beings. Typically, the active compounds for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the active compound is to be administered by infusion, it can be dispensed, for example, with an infusion bottle containing sterile pharmaceutical grade water or saline. Where the active compound is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an orally administered of the active compound. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the active compound can be delayed or prolonged by proper formulation. For example, a slowly soluble pellet of the active compound can be prepared and incorporated in a tablet or capsule. The technique can be improved by making pellets of several different dissolution rates and filling capsules with a mixture of the pellets. Tablets or capsules can be coated with a film that resists dissolution for a predictable period of time. Even the parenteral preparations can be made long acting, by dissolving or suspending the compound in oily or emulsified vehicles, which allow it to disperse only slowly in the serum.

Compositions for use in accordance with the present invention can be formulated in conventional manner using one or more physiologically acceptable carriers or excipients.

Thus, the compound and optionally another therapeutic or prophylactic agent and their physiologically acceptable salts and solvates can be formulated into pharmaceutical compositions for administration by inhalation or insufflation (either through the mouth or the nose) or oral, parenteral or mucosol (such as buccal, vaginal, rectal, sublingual) administration. In some embodiments, the administration is optical (e.g. eyes drops applied directly to the eye). In one embodiment, local or systemic parenteral administration is used.

For oral administration, the compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician's Desk Reference (62^{nd} ed. 2008, herein incorporated by reference in its entirety).

Methods of administering the active compound and optionally another therapeutic or prophylactic agent include, but are not limited to, parenteral administration (e.g., intradermal, intramuscular, intraperitoneal, intravenous and subcutaneous), epidural, and mucosal (e.g., intranasal, rectal, vaginal, sublingual, buccal or oral routes). In a specific embodiment, the active compound and optionally another prophylactic or therapeutic agents are administered intramuscularly, intravenously, or subcutaneously. The active compound and optionally another prophylactic or therapeutic agent can also be administered by infusion or bolus injection and can be administered together with other biologically active agents. Administration can be local or systemic. The active compound and optionally the prophylactic or therapeutic agent and their physiologically acceptable salts and solvates can also be administered by inhalation or insufflation (either through the mouth or the nose). In a preferred embodiment, local or systemic parenteral administration is used.

In specific embodiments, it can be desirable to administer the active compound locally to the area in need of treatment. This can be achieved, for example, and not by way of limitation, by local infusion during surgery or topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the active compound can be formulated as a suppository, with traditional binders and vehicles such as triglycerides.

The amount of the active compound that is effective in the treatment or prevention of macular degeneration or angiogenesis can be determined by standard research techniques. For example, the dosage of the active compound which will be effective in the treatment or prevention of age-related macular degeneration can be determined by administering the active compound to an animal in a model such as, e.g., the animal models known to those skilled in the art. In addition, in vitro assays can optionally be employed to help identify optimal dosage ranges.

Selection of a particular effective dose can be determined (e.g., via clinical trials) by a skilled artisan based upon the consideration of several factors, which will be known to one skilled in the art. Such factors include the disease to be treated or prevented, the symptoms involved, the subject's body mass, the subject's immune status and other factors known by the skilled artisan.

The dose of the active compound to be administered to a subject, such as a human, is rather widely variable and can be subject to independent judgment. It is often practical to administer the daily dose of the active compound at various hours of the day. However, in any given case, the amount of the active compound administered will depend on such factors as the solubility of the active component, the formulation used, subject condition (such as weight), and/or the route of administration.

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); and BSA (bovine serum albumin).

### Example I

### Materials and Methods

Cell Lines, Cultures and Reagents

Human umbilical vein endothelial cells (HUVEC) were kindly gifted from Dr. Xinli Wang (Cardiothoracic Surgery Division, Michael E. DeBakey Department of Surgery, Baylor College of Medicine Hospital). The human prostate cancer cell line (PC3) was purchased from the American Type Culture Collection (Manassas, Va.) and maintained in a mixture of RPMI-1460 medium and 5% fetal bovine serum. HTScan® VEGF receptor 2 kinase assay kit was ordered from Cell Signaling Technology. HRP labeled secondary antibody, TMB substrate and stop solution were kindly gifted by Cell Signaling Technology. Streptavidin coated yellow 96-well plates were kindly gifted by PerkinElmer Life Sciences. Matrigel was ordered from BD Biosciences, Bedford, Mass.

### Proliferation Assay

HUVEC and PC3 cell proliferation assays with different concentrations of GA were followed according to the CellTiter 96 Aqueous One Solution Cell Proliferation Assay (Promega) and analyzed using a VERSAMAX microplate reader (Molecular Devices).

### Flow Cytometry and FACS Analysis

About 2×10⁶ HUVEC and PC3 cells were treated with different concentrations of GA at 37° C. in a 5% CO₂ incubator for 24 hours. The cells were collected and flow cytometry was performed using a FACS Vantage SE DiVa flow cytometer (Becton Dickinson) with propidium iodide staining. The cell population percentages at Sub G1 were defined as apoptotic cells.

### Migration Assay

HUVEC cells were allowed to grow to full confluence on six-well plates precoated with 0.1% gelatin. Monolayer cells were wounded by scratching with 1 ml pipette tip and washed three times with 1×PBS. Fresh endothelial cell growth medium (hereinafter, ECGM) was added with or without 4 ng/ml VEGF, which was received from the NIH experimental branch, and supplemented with different concentrations of GA. Images were taken after 24 hours of incubation at 37° C. in a 5% CO₂ incubator (hereinafter, 5% CO₂) with a Nikon digital camera. For PC3 cell migration, PC3 cells were allowed to grow to full confluence on six-well plates and wounded by scratching with a 1 ml pipette tip and washed three times of 1×PBS. Fresh 1640 medium was added with 4 ng/ml VEGF and different concentrations of GA. After a 24-hour incubation period, images were take with a Leica DM IRB inverted microscope. The migrated cells were quantified by manual counting of high power fields (HPF, 200×) and percent inhibition was expressed using untreated wells as 100% (t-test, p<0.005). Similar patterns of the inhibition effects were observed in three independent experiments.

### Transwell Migration Assay

The transwell (Corning Incorporated, NY, USA) were coated with 0.1% gelatin (Sigma) for 30 minutes at 37° C. After washed the transwells three times with 1×PBS, the bottom chambers (600 μl) were filled with ECGM medium with 20% FBS supplemented with 4 ng/ml VEGF and the top chambers were seeded with 100 μl ECGM medium and HUVEC cells (4×10⁴ cell/well). The top and bottom chambers contained the same series of concentrations of gambogic acid (GA). HUVEC cells were allowed to migrate for 4 hours at 37° C., 5% CO₂. After the incubation, cells on the top surface of the membrane (nonmigrated) were scraped with a cotton swab. Cells on the bottom side of the membrane (migrated cells) were fixed with 4% paraformaldehyde for 20 minutes and washed three times with 1×PBS. The cells were stained by hematoxylin and eosin (hereinafter, H&E) staining and then destained with 1×PBS (pH 7.4). The membranes were left to air dry at room temperature for 30 minutes. Images were taken using an OLYMPUS inverted microscope and migrated cells were quantified by manual counting using high power fields (HPF, 200×). The invaded cells were calculated and percent inhibition was expressed using untreated wells as 100% (t-test, p<0.01).

### Tubes Formation Assay

Matrigel (BD Biosciences) were thawed at 4° C. overnight and each well of the prechilled 24-well plates was coated with 100 μl Matrigel and incubated at 37° C. for 45 minutes. HUVEC cells (4×10⁴ cells) were added in 1 ml ECGM with various concentrations of gambogic acid. After 12-16 hours of incubation at 37° C., 5% CO₂, endothelial cell tube formation was assessed using an OLYMPUS inverted microscope. Tubular structures were quantified by manual counting of low power fields (25×) and percent inhibition was expressed using untreated wells as 100% (t-test, p<0.001).

### Aortic Ring Assay

Forty-eight-well plates were covered with 100 μl of Matrigel at 4° C. and incubated at 37° C., 5% CO₂ for 30 minutes. Aortas isolated from mice were cleaned of periadventitial fat and connective tissues, and cut into about 1-1.5 mm-long rings. After being rinsed five times with endothelial cell-based medium, the aortas were placed on the Matrigel covered wells and covered with another 100 μl of Matrigel. Artery rings were cultured in 1.5 ml of ECGM medium without serum for 24 hours. The medium was then replaced with 1.5 ml of ECGM medium supplemented with or without GA. The medium was changed every two days with the exact composition as described above. After 4 days incubation, the micro-vessel growth was quantified in the mouse aortic ring assay by taking photographs with an Olympus IX 70 inverted microscope using a 4× objective lens. After images were acquired, the outgrowth area was delineated and measured with Pro Plus software (Media Cybernetics).

### Matrigel Plug Assay

Matrigel (0.5 ml/plug) with no VEGF or GA, VEGF (4 ng/ml) but no GA, VEGF (4 ng/ml) and 0.1 μM GA or 0.2 μM GA in liquid form at 4° C., respectively, were injected subcutaneously in the midventral abdominal region of 5-6 week old C57BL/6 mice (five mice for each group). After 7 days, the mice were sacrificed and the plugs were removed. Each concentration had 4-5 Matrigel plugs. The Matrigel plugs were fixed with formalin and embedded with paraffin. The 5 μm sections were stained with H&E staining. The number of erythrocyte-filled blood vessels in the high power microscope field (HPF, 200×) was recorded (plug number=4-5, t-test, p<0.005).

### Xenograft Mouse Model

The 5-6 week old severe combined immune deficiency (SCID) male mice (ordered from NIH, each weighing about 20 g) were divided into groups of five mice per group. Log-phase PC3 human prostate tumor cells were subcutaneously injected (2×10⁶ cell per mouse) into the mice. After the tumors had become established (about 50 mm³), the mice were subcutaneously injected with or without 3 mg/kg GA every day. The mice body weights and tumor sizes were recorded every day and the tumor sizes were determined by Vernier caliper measurements and calculated as length×width×height. After 15 days, mice with subcutaneous (hereinafter, s.c.) tumors no greater than 1.5 cm in diameter were sacrificed in accordance with University of California Los Angeles Animal Rights Committee Guidelines.

### Histology and Immunohistochemistry

The tumors were removed and fixed with Histochoice® MB (Molecular Biology) tissue fixative (Amresco®) and embedded with paraffin. The 5 μm sections were performed specific blood vessel staining with CHEMICON's Blood Vessel Staining Kit (von Willebrand Factor, Chemicon International, blood vessel staining kit, peroxidase system). Images were taken with a ZEISS Axioskop 40 photomicroscope. The number of blood vessels in the high power fields (HPF, 200×) was counted (plug number=4-5, t-test, p<0.005).

### VEGF Receptor 2 Inhibition Assay

12.5 μl of the 4× reaction cocktail containing 100 ng VEGF Receptor 2 (supplied from the HTScan® VEGF receptor 2 kinase assay kit, Cell Signaling Technology, USA) was incubated with 12.5 μl/tube of GA for 5 minutes at room temperature. 25 μl of 2×ATP/substrate peptide cocktail was added to the pre-incubated reaction cocktail/GA compound. After incubation at room temperature for 30 minutes, a 50 μl/tube stop buffer (50 mM EDTA, pH 8) was added to each tube to stop the reaction. Then 25 μl of each reaction was transferred with 75 μl H₂O/well to a 96-well streptavidin-coated plate (PerkinElmer Life Sciences, USA) and incubated at room temperature for 60 minutes. After washing three times with 200 μl/well PBS/T (0.05% Tween-20 in 1×PBS), a 100 μl primary antibody (Phosphor-Tyrosine Monoclonal Antibody (P-Tyr-100), 1:1000 in PBS/T with 1% BSA) was added per well. After incubation at room temperature for 60 minutes, the wells were washed three times with 200 μl PBS/T. A 100 μl diluted HRP labeled anti-mouse IgG (1:500 in PBS/T with 1% BSA) was added per well. After incubation at room temperature for 30 minutes, the wells were washed five times with 200 μl PBS/T per well. Then a 100 μl/well TMB substrate was added per well and the plate was incubated at room temperature for 15 minutes. The stop solution (100 μl/well) was added and mixed followed incubation at room temperature 15 minutes. The plate was then detected at 405 nm with VERSAMAX microplate reader (Molecular Devices). The assay (mean±SEM, n=3) was repeated 3 times.

### Western Immunoblotting

HUVEC cells pretreated with or without 4 nM VEGF for 5 min were treated with or without different concentrations of GA for another 5 minutes. 200 μg total protein of the cells of each sample was subjected to immunoprecipitation using anti-c-Src, anti-FAK and anti-AKT antibodies (Santa Cruz Biotech) and further subjected to Western blotting. The pTyr-antibody (Santa Cruz Biotech) was used for detecting c-Src phosphorylation and the pFAK397 antibody (Cell Signaling) was blotted for c-Src-associated FAK phosphorylation at multiple tyrosine residues. In addition, AKT phosphorylation was examined using a pSer473-AKT antibody (Cell Signaling). Anti-cleaved caspase 3 antibody (Santa Cruz Biotech) was used for detecting cleaved caspase 3 and polyADP ribose polymerase (PARP) cleavage was detected by an anti-PARP p85 fragment (Promega) in apoptosis assays.

### Statistical Analysis

The data (mean±SEM, n=3) were analyzed following three rounds of cell proliferation, apoptosis, migration, invasion, and aortic ring assays. Statistical significance of differences between control and sample groups was determined by using the t-test. The minimal level of significance was P<0.05.

### Results

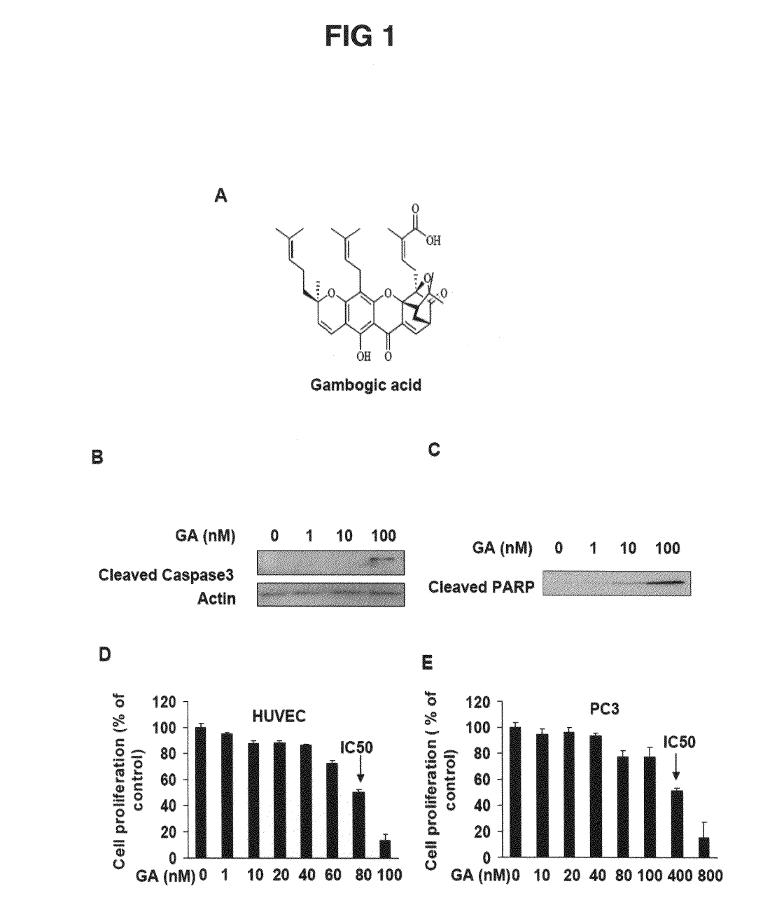

Several previous studies described gambogic acid (FIG. 1A) activated apoptosis in cancer cells, which partly answered the molecular mechanism of GA's anticancer characters. Since angiogenesis is crucial for tumor growth and metastasis and endothelial cells play key roles in angiogenesis, we first examine if GA also promotes apoptosis in human vascular endothelial cells (HUVECs). Whole cell proteins of GA treated HUVECs were analyzed by Western blotting with anticleaved caspase 3 and PARP antibodies. The cleaved caspase 3 (FIG. 1B) and PARP (FIG. 1C) assays indicated GA strongly induced apoptosis at 100 nM in HUVEC cells. To investigate if the apoptotic activation effect of GA is different between endothelial cells and cancer cells, we measured the apoptotic populations of HUVECs and human prostate cancer cell PC3 cells treated with GA by flow cytometry FACS assays. We found that GA at 80 nM induced apoptosis in only 4% of PC3 cells while in HUVEC cells GA induced 40% of the cells into apoptosis (Table 1), indicating GA was much more effective in promoting cell apoptosis for endothelial cells versus cancer cells. It has been demonstrated that GA inhibited human hepatoma SMMC-7721 proliferation. To determine the effects of GA on HUVEC cell proliferation, we performed proliferation assays of HUVEC cells using different concentrations of GA and compared their effects with PC3 cancer cell assays. We found that 80 nM GA inhibited HUVEC cell proliferation by 50% (FIG. 1D) while more than 400 nM GA is required to obtain the same inhibitory effect in PC3 cancer cells (FIG. 1E), indicating that GA was more effective in inhibiting endothelial cell proliferation than cancer cell proliferation.

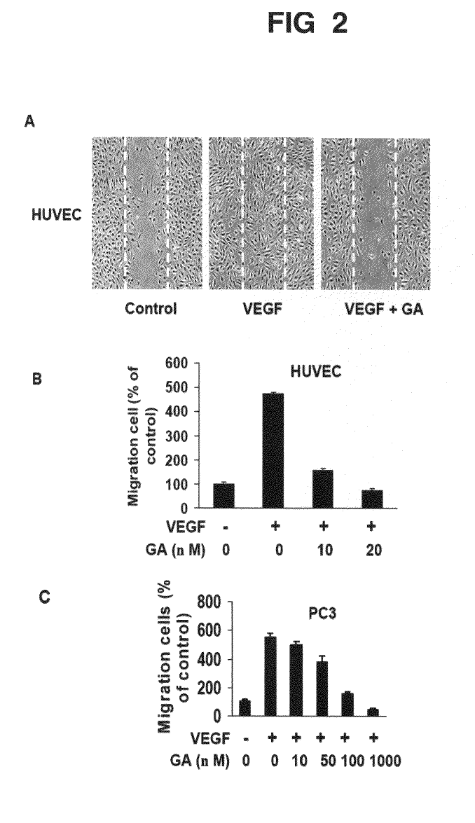

As cell migration is necessary for endothelial cells in both angiogenesis and cancer cell related tumor growth and metastasis, we performed wound-migration assays to determine GA's affects on the migration of both HUVEC cells and PC3 cells. We found that 10 nM GA strongly inhibited VEGF dependent migration of HUVEC cells (FIGS. 2A and B), while more than 100 nM GA was required to inhibit VEGF dependent migration for PC3 cancer cells (FIG. 2C), indicating that GA suppressed VEGF-induced cell migration for both endothelial cells and cancer cells and that endothelial cells are more sensitive to GA inhibition as compared to cancer cells.

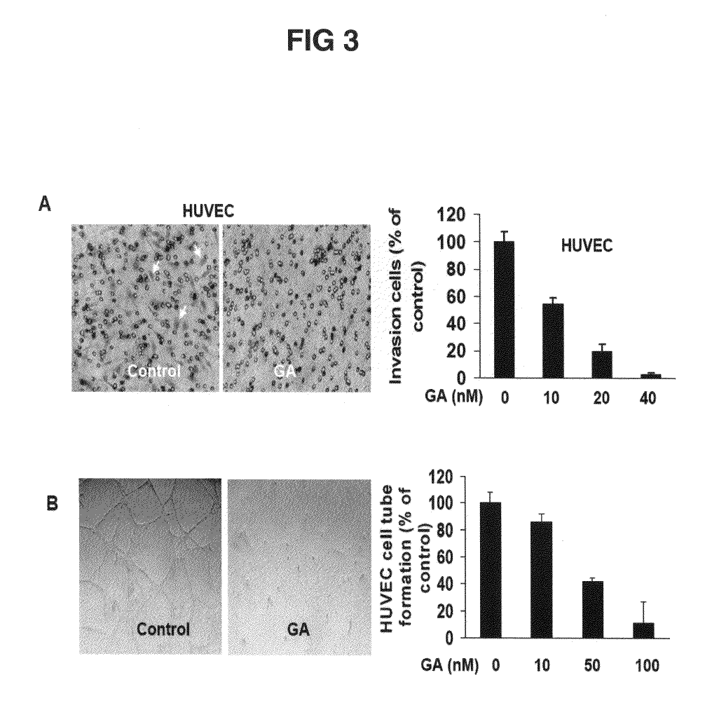

Endothelial cell invasion is necessary for angiogenesis. To evaluate the effect of GA on endothelial cell invasion, we performed transwell assays and found that 40 nM GA inhibited almost all invasion activities of HUVEC cells (FIG. 3A), suggesting that GA significantly inhibited the invasion properties of endothelial cells at very low concentrations (nM). Although angiogenesis is a complex procedure involving several kinds of cells, tube formation by endothelial cells is the key step. To further investigate the effect of GA on endothelial cell tube formation, we added HUVEC cells (4×10⁴ cells) in 1 ml ECGM with different concentrations of GA onto Matrigel layers. After 12-16 hours of incubation, the ability of endothelial cells to form tube-like structures was assessed with an inverted photomicroscope. Approximately 50 nM GA inhibited tube formation of HUVEC cells by 50% on Matrigel based assays while 100 nM GA completely inhibited the tube-forming ability of HUVECs on Matrigel (FIG. 3B).

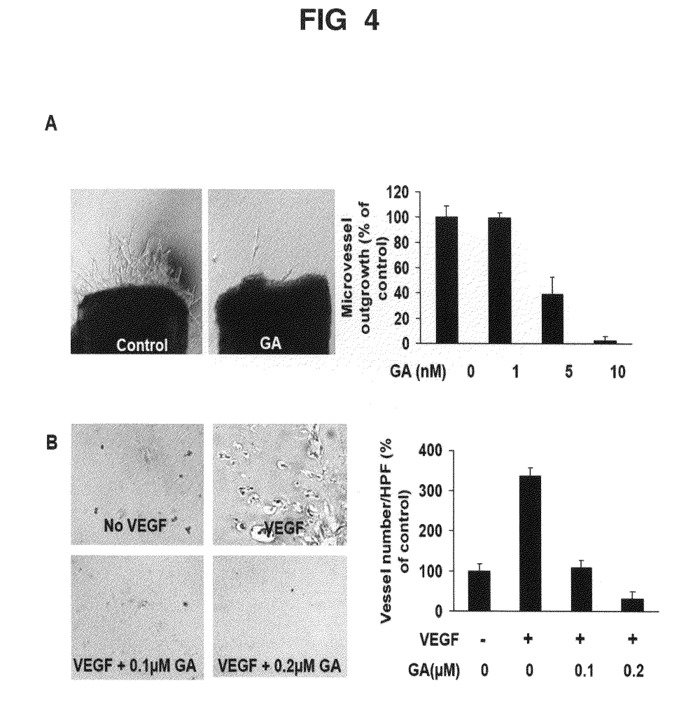

To examine the inhibitory effect of GA on angiogenesis, we performed aortic ring assays using isolated aortas from mice. The 1-1.5 mm-long aortic rings were put on Matrigel and covered by another Matrigel layer and ECGM medium with or without GA. After 4 days of incubation for the aortic rings, micro-vessel growth was quantified and compared in the presence or absence of different concentrations of GA. We found that GA at 10 nM (higher concentration not shown) inhibited almost all new vessel growth (FIG. 4A), suggesting GA dramatically inhibited angiogenesis in vitro. To further verify the inhibitory effect of GA on angiogenesis, we used Matrigel plug assays to examine the anti-angiogenesis effect of GA in vivo. We subcutaneously injected Matrigel (0.5 ml/plug) with no VEGF, VEGF (4 ng/ml), VEGF (4 ng/ml) and 0.1 μM GA or 0.2 μM GA in the midventral abdominal region of 5-6 week old C57BL/6 mice (five mice for each group). After 7 days, the mice were sacrificed and the Matrigel plugs were removed, sectioned, and H&E stained. As shown in FIG. 4B, 0.1 μM GA inhibited VEGF dependent angiogenesis while 0.2 μM GA totally abolished angiogenesis in the Matrigel plug assays (FIG. 4B), indicating GA inhibited angiogenesis in vivo. Based on the above analyses, we concluded that GA inhibited angiogenesis in vitro and in vivo using different assays.

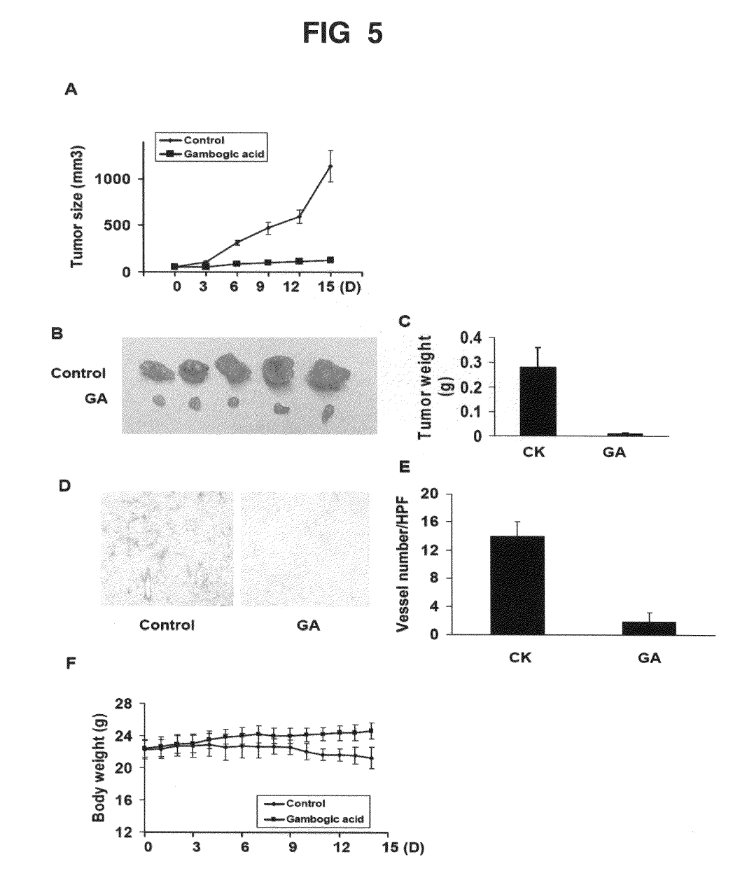

Tumor angiogenesis provides oxygen, nutrients and main routes for tumor growth and metastasis and acts as a rate-limiting step in tumor propagation. To determine the effect of GA on tumor angiogenesis and tumor growth, we injected s.c. (2×10⁶ PC3 cell per mouse) into the mice. It has been demonstrated that 4 mg/kg of GA at a frequency of 1 treatment every two days is a non-toxic dosage. After the tumors had become established (about 50 mm³), the mice were subcutaneously injected with or without 3 mg/kg GA every day. The mouse body weights and tumor sizes were recorded every day and the tumor sizes were measured by Vernier calipers and calculated as length×width×height as previous described. After 15 days, the mice were sacrificed and the tumors were removed. As shown in FIG. 5A, at day 15 after injection of tumor cells, the average tumor size of control group was 1144±169 mm³ while that of GA treated group was 169.1±25.6 mm³ (FIGS. 5A and 5B). The average tumor weight of control was 0.28±0.08 g while that of GA treated group was 0.072±0.0008 g (FIGS. 5B and 5C), indicating GA significantly inhibited tumor growth. To examine the inhibitory effect of GA on tumor angiogenesis, we stained the 5 μm tumor sections with a blood vessel specific staining kit (FIG. 5D). The average vessel number in tumors of the control group was 14±2 (HPF) while that in the GA treated group was 1.8±1.3 (HPF) (FIG. 5E), suggesting GA significantly inhibited tumor angiogenesis and prevented tumor growth. To evaluate the side effect or chemotoxicity of GA on normal growth in mice, we recorded the body weights of the mice everyday. During the 15 day period, the average body weight of the control group decreased 1±1.3 g while that of GA treated group increased 3.2±0.9 g (FIG. 5F), indicating that 3 mg/kg GA for mice everyday may be a non-toxic dosage or at least a low toxic dosage.

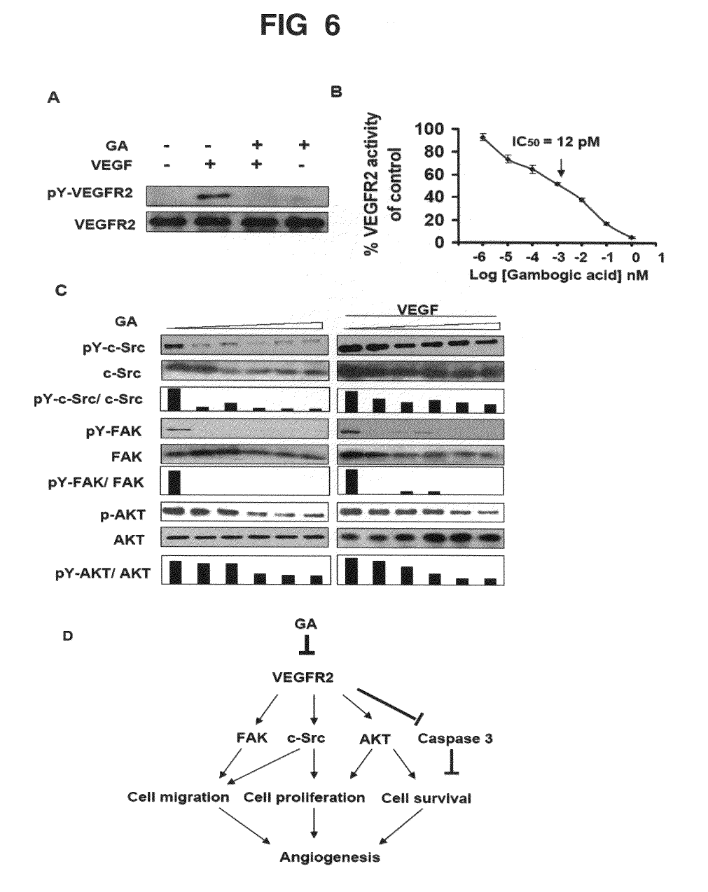

VEGFR2 is the primary receptor in VEGF signaling pathway that regulates endothelial cell proliferation, migration, differentiation, tube formation, and angiogenesis. In order to explore the molecular mechanism of GA's anti-angiogenesis, we examined whether GA inhibits the activation of VEGFR2. We first examined the phosphorylation and activation of VEGFR2 with or without GA. HUVEC cells were stimulated with VEGF; phosphorylation and activation of VEGFR2 was detected by immunoprecipitation with anti-VEGFR2 (Flk-1) antibody and by Western blotting with anti-pTyr-antibody for phosphorylation of VEGFR2. Treatment of GA dramatically inhibited the phosphorylation of VEGFR2 in the assays (FIG. 6A), suggesting GA is a potential inhibitor of VEGFR2. To verify the inhibitory effect of GA on VEGFR2, we further examined the effects of GA on specific activation of VEGFR2 with the HTScan® VEGFR2 kinase assay kit according to the manufacturer's suggested methods (Cell Signaling Technology and PerkinElmer Life Sciences, USA). We found GA inhibited VEGFR2 kinase activity with an IC₅₀ value of 12 pM (FIG. 6B). Together, these data indicate that GA is a VEGFR2 inhibitor.

VEGFR2 regulates focal adhesion turnover during cell migration by mediating the activation of focal adhesion kinase (FAK) and its substrate of paxillin. Upon stimulation, VEGFR2 promotes the activation c-Src and mediates cell migration and proliferation. To understand the inhibitory effects of GA on cell proliferation and migration, we directly measured how GA regulates the phosphorylation and activation of Src and FAK. Whole cell proteins from HUVEC cells were treated with or without VEGF. The proteins were isolated by immunoprecipitation with anti-c-Src and FAK antibodies and Western blotting was performed with the anti-pFAK antibody for FAK phosphorylation and the anti-pTyr-antibody for the activation of c-Src, respectively. GA significantly inhibited the phosphorylation and activation of both c-Src and FAK but not c-Src or FAK protein expression (FIG. 6C), suggesting GA inhibited cell migration and proliferation by blocking VEGFR2 and arresting its downstream effects. The activation of c-Src with VEGF pretreatment (FIG. 6C, left) was stronger than without VEGF treatment (FIG. 6C, right), which was consistent with VEGF promoted c-Src activation by stimulation of VEGFR2.

The phosphorylation of Y1175 of VEGFR2 mediates the activation of AKT for regulating cell proliferation. To further examine the downstream signaling pathways mediated by VEGFR2, we examined the activation of the serine/threonine kinase AKT (PKB) with or without the treatment of GA using anti-pSer473-AKT antibody. HUVEC cells were treated with or without VEGF and whole cell proteins were analyzed by immunoprecipitation with anti-AKT antibody and Western blotting with anti-pSer473 antibody for the phosphorylation and activation of AKT. As shown in FIG. 6C, GA inhibited AKT phosphorylation and activation with increasing concentration of the GA in the absence (FIG. 6C, left) or presence of VEGF (FIG. 6C, right), suggesting GA inhibited cell proliferation by regulating the activation of AKT signaling pathways.

### Discussion

We identified GA as a VEGF receptor 2 inhibitor and comprehensively demonstrated that GA inhibited angiogenesis and tumor progression. Our work focuses on GA's inhibitory effects on HUVEC cell proliferation, migration, invasion, and tube formation, four key characteristics of endothelial cells in angiogenesis. By directly blocking VEGFR2 phosphorylation and activation, GA suppressed the AKT signaling pathway and inhibited cellular proliferation. At the same time, GA inhibited the phosphorylation and activation of Src and FAK, key protein kinases in cell migration and adhesion signaling pathways. GA significantly inhibited angiogenesis both in vitro and in vivo. Using non-toxic dosages of GA, we demonstrated that GA could inhibit tumor angiogenesis and tumor growth in SCID mouse models.

In this study, we showed previously unreported inhibitory effects of GA on angiogenesis both in vitro and in vivo (FIG. 4) and discovered that GA is a potent VEGFR2 inhibitor (FIG. 6), a new role for xanthone family members. As compounds that act as RTK inhibitors always show inhibitory characteristics for multiple kinases, we will investigate whether GA can inhibit other receptor tyrosine kinases such as VEGFR1, VEGFR3, FGFRs, PDGFRs in our future studies. The activity of VEGFR2 is very well controlled in normal cells; mutations or overexpression of VEGFR2 induces abnormal downstream activities, leading to many human cancers. We discovered that GA was more sensitive to HUVEC cells as compared to PC3 cancer cells in apoptosis activation (Table 1), inhibitory effects on cell proliferation (FIGS. 1D and 1E) and migration (FIGS. 2B and 2C), which was consistent with previous studies. We showed GA significantly inhibited angiogenesis (FIG. 4) and tumor angiogenesis (FIGS. 5D and 5E) together with tumor growth prevention (FIGS. 5A, 5B and 5C), which matched well with the known concept that angiogenesis is a rate limiting step for tumor growth. These findings may supply molecular mechanism clues for GA as a new anti-angiogenesis candidate with low chemotoxicity, which is very important for clinical usage.

Phosphorylation of VEGFR2's Tyr-1175 is required for binding and activation of Shb and phosphoinositide-3 kinase (PI3K), which is critical for subsequent activation of AKT and endothelial cell proliferation. We found that GA inhibited the activation of VEGFR2 (FIGS. 6A and 6B) and AKT (FIG. 6C) as well as cell proliferation (FIGS. 1D and 1E). Phosphorylation of Tyr-951 of VEGFR2 is required for the binding site of TSAD, which mediates its substrate of c-Src and then regulates cell migration. VEGFR2 also directly regulates the phosphorylation of FAK and mediates cell migration. Here we showed that GA inhibited the activation of FAK and c-Src (FIG. 6C) and cell migration (FIG. 2). It has been demonstrated that GA promotes tumor necrosis factor (TNF) induced apoptosis and inhibits TNF-induced cellular invasion through the NF-κβ signaling pathway. TNF, via its receptor 2 (tumor necrosis factor receptor 2, TNFR2), regulates endothelial cell migration, invasion and tube formation, but TNFR2 lacks intrinsic kinase activity. Interestingly, Zhang R et al. reported that the activation of TNFR2 was VEGFR2 dependent and that VEGFR2 inhibitor suppressed the activation of VEGFR2 and then inhibited TNFR2's activation. We found that GA could function as a VEGFR2 inhibitor (FIGS. 6A and 6B), significantly blocking endothelial cell migration, invasion, and tube formation. Thus, it is possible that GA regulates NF-κβ signaling by inhibiting the activation of VEGFR2 and TNFR2.

In summary, our studies demonstrate an axis of action by GA, e.g. GA functions as an inhibitor of VEGFR2 and its signaling pathway, leading to the inhibition of angiogenesis and tumorigenesis (FIG. 6D). We demonstrated the previously unreported inhibition of GA on HUVEC cell proliferation, migration, and tube formation, as well as the anti-angiogenesis activity of GA in vitro and in vivo. Our data suggest the potential for compounds of the xanthone family as anti-angiogenesis and anti-cancer drugs.

TABLE I
Table 1. GA activates apoptosis on PC3 and HUVEC cells.

| | Apoptotic population (% of total) | | | | |
|---|---|---|---|---|---|
| GA (nM) | 0 | 20 | 40 | 80 | 100 |
| PC3 | 1 ± 0.3 | 1.6 ± 0.3 | 2.9 ± 0.4 | 4.3 ± 0.5 | 13.8 ± 3.6 |
| HUVEC | 2.3 ± 0.2 | 11.6 ± 1.1 | 14.4 ± 2.3 | 40 ± 3.6 | 57.6 ± 4.7 |

We claim:

1. A method of treating macular degeneration comprising:
a) providing:
i) a subject diagnosed with macular degeneration, and
ii) a composition comprising gambogic acid;
b) administering said compound to said subject.

2. The method of claim 1, wherein said subject is a mammal.

3. The method of claim 1, wherein said composition is a solution.

4. The method of claim 1, wherein the mode of said administration is selected from the group consisting of optical, oral, parenteral, mucosol, buccal, vaginal, rectal, sublingual, inhalation, insufflation, intravenous, intrathecal, subcutaneous and intramuscular.

5. A method of treating angiogenesis comprising:
a) providing:
i) a subject diagnosed with or at risk for angiogenesis, and
ii) a composition comprising gambogic acid;
b) administering said compound to said subject.

6. The method of claim 5, wherein said subject is a mammal.

7. The method of claim 5, wherein said composition is a solution.

8. The method of claim 5, wherein the mode of said administration is selected from the group consisting of optical, oral, parenteral, mucosol, buccal, vaginal, rectal, sublingual, inhalation, insufflation, intravenous, intrathecal, subcutaneous and intramuscular.

9. A method for treating a disease characterized by angiogenesis comprising:
a) providing:
i) a subject diagnosed with or at risk for said disease characterized by angiogenesis, and
ii) a composition comprising gambogic acid;
b) administering said compound to said subject.

10. The method of claim 9, wherein said subject is a mammal.

11. The method of claim 9, wherein said composition is a solution.

12. The method of claim 9, wherein the mode of said administration is selected from the group consisting of optical, oral, parenteral, mucosol, buccal, vaginal, rectal, sublingual, inhalation, insufflation, intravenous, intrathecal, subcutaneous and intramuscular.

13. The method of claim 9, wherein said disease is selected from the group consisting of corneal angiogenesis, diabetic retinopathy, inflammation, rheumatoid arthritis, psoriasis and impaired wound healing.

14. A method of treating macular degeneration in a mammal comprising:
a) providing:
i) a mammal exhibiting symptoms associated with macular degeneration, and
ii) a composition comprising gambogic acid;
b) administering said composition to said mammal under conditions such that said symptoms are reduced.

15. The method of claim 14, wherein said composition is a solution.

16. The method of claim 14, wherein the mode of said administration is selected from the group consisting of optical, oral, parenteral, mucosol, buccal, vaginal, rectal, sublingual, inhalation, insufflation, intravenous, intrathecal, subcutaneous and intramuscular.

17. A method for treating a disease characterized by angiogenesis comprising:
a) providing:
i) a mammal exhibiting symptoms associated with said disease, and
ii) a composition comprising gambogic acid or a gambogic acid derivative;
b) administering said composition to said mammal under conditions such that said symptoms are reduced.

18. The method of claim 17, wherein said subject is a mammal.

19. The method of claim 17, wherein said composition is a solution.

20. The method of claim 17, wherein the mode of said administration is selected from the group consisting of optical, oral, parenteral, mucosol, buccal, vaginal, rectal, sublingual, inhalation, insufflation, intravenous, intrathecal, subcutaneous and intramuscular.

21. The method of claim 20, wherein said disease is selected from the group consisting of cornea angiogenesis, diabetic retinopathy, inflammation, rheumatoid arthritis, psoriasis and impaired wound healing.
